(19) United States
(12) Patent Application Publication
(10) Pub. No.: US 2025/0152383 A1
(43) Pub. Date: May 15, 2025

(54) PROSTHETIC GLOVE AND METHOD FOR PRODUCING SAME

(71) Applicant: OTTO BOCK HEALTHCARE PRODUCTS GMBH, Wien (AT)

(72) Inventors: Walter LUNZER, Wien (AT); Sonja WAGNER, Wien (AT); Angelika WILLCOCK, Wien (AT)

(21) Appl. No.: 18/837,772

(22) PCT Filed: Mar. 2, 2023
(86) PCT No.: PCT/EP2023/055319

§ 371 Date: Aug. 12, 2024

(30) Foreign Application Priority Data
Mar. 16, 2022 (DE) 10 2022 106 213.9

(51) Int. Cl. A61F 2/50; A61F 2/58; A61F 2/78

(52) U.S. Cl. CPC A61F 2/5044; A61F 2/586; A61F 2/7812

(57) ABSTRACT

The invention relates to a prosthetic glove and to a method for producing same, having the steps of providing a first textile blank (10) with a hand rear surface (11) and finger sections (12, 13, 14, 15, 16) extending away from the hand rear surface (11), providing a second textile blank (20) (20) with a hand inner surface (21) and finger sections (22, 23, 24, 25, 26) extending away from the hand inner surface (21), wherein at least the finger sections (12, 13, 14, 15, 16, 22, 23, 24, 25, 26) of the textile blanks (10, 20) are designed to be elastic or have elastic regions, arranging the two textile blanks (10, 20) such that correspondingly designed finger sections (12, 13, 14, 15, 16, 22, 23, 24, 25, 26) are paired together, and connecting the two textile blanks (10, 20) directly together along the longitudinal extension of the finger sections (12, 13, 14, 15, 16, 22, 23, 24, 25, 26) at the edge regions thereof in order to form finger sleeves (50). In a relaxed state, at least one finger section (22, 23, 24, 25, 26) of the second textile blank (20) is shorter than the opposing paired finger section (12, 13, 14, 15, 16) of the first textile blank (10) and is stretched upon connecting the finger section (22, 23, 24, 25, 26) of the second textile blank (20).

The invention relates to a method for producing a prosthetic glove comprising the steps of providing a first textile blank having a dorsum of hand surface and finger portions that extend away from the dorsum of hand surface; providing a second textile blank having a palm of hand surface and finger portions that extend away from the palm of hand surface, wherein the finger portions of the textile blanks are of an elastic design or comprise elastic regions; arranging the two textile blanks in such a manner that correspondingly designed finger portions are assigned to one another, in particular lie opposite one another; and connecting the two textile blanks directly to one another along the longitudinal extent of the finger portions on the peripheral regions of the latter so as to form finger sleeves. The invention likewise relates to a prosthetic glove having a first textile blank having a dorsum of hand surface and finger portions that extend away from the dorsum of hand surface, a second textile blank having a palm of hand surface and finger portions that extend away from the palm of hand surface, wherein at least the finger portions of the textile blanks are of an elastic design or comprise elastic portions, and correspondingly designed finger portions lie opposite one another.

Prosthetic hands are complex mechanical devices which are often motorized and enable a user with an inexistent or no longer existent hand to perform activities and functions which make everyday life easier and enhance the quality of life. This includes in particular gripping, holding and releasing objects. In order for such functions and activities to be enabled, prosthetic fingers are mounted so as to be articulated on a main body that corresponds to the palm of a natural hand. These prosthetic fingers per se can be of an articulated design and are provided with or coupled to drives in such a way that different gripping positions can be assumed. In order for the complex and to some extent sensitive mechanism, electrics and electronics to be protected, prosthetic hands are provided with a cover, a so-called prosthetic masking, which is in particular designed as a prosthetic glove. The prosthetic glove protects firstly the prosthetic hand, and secondly shields the user from the mechanical components of the prosthetic hand. Moreover, the prosthetic glove can offer a visual cladding in the mechanical components in such a way that the prosthetic hand can be used in an ideally unobtrusive manner.

Prosthetic gloves can be designed using different materials; prosthetic gloves made of an elastomer material, for example silicone, are known to provide an ideally natural visual appeal. Such prosthetic gloves are produced by a dipping method or cast in a mold. The advantages of such a prosthetic masking lie in the compact surface offering a good protection in relation to moisture, as well as in the visual appearance and surface characteristics which come close to the natural appearance. The high weight, and a high resistance to deformation due to wrinkling when bending the prosthetic fingers, are problematic.

Foam materials, for example so-called neoprene, which are lighter than elastomers but still have a large material thickness and as a result have a tendency toward wrinkling, are also used as an alternative to an elastomer.

Furthermore known are prosthetic gloves or prosthetic maskings which are largely composed of a textile material. One possibility for producing such a glove lies in producing the latter integrally by a knitting method. This is comparatively complex and not lucrative for the producing knitting industry in view of the relatively minor production volume of prosthetic gloves which are specially adapted in terms of shape and size. Furthermore, according to the current prior art, such fine knitwear cannot yet be produced based on the fully-fashioned technology in such a way that a surface is produced which would be sufficiently smooth in order to generate haptics which are adequately similar to skin. An alternative lies in sewing a prosthetic glove from individual blanks. For maximum mobility of the fingers, the classic glove patterns provide a separately inserted thumb blank in the thumb region, and the so-called finger walls, which are designed as strips between two fingers, for the region between the fingers. Manufacturing such a prosthetic glove is complex and expensive due to the many blanks and the seams to be sewn; moreover the seam allowances required for stitching add bulk in particular at the fingertips. This generates a disadvantageous visual effect, and moreover also leads to unevenness at the fingertips, which has a negative influence on the gripping surface of the fingertips.

A further form of a pattern of a textile glove is composed of two identical blanks in the shape of a hand, which are stitched to one another. In the process, the seams which are guided circumferentially about the blanks lie between the fingers, which may block the converging movement of the fingers when gripping, because prosthetic fingers cannot yet be spread apart like the human hand. Therefore, seam allowances that cause bulking or thickening between two fingers are to be avoided. Moreover, a greater gripping force to be applied is required when closing the prosthetic hand in the region of the upper side of the hand, or of the dorsum of the hand, in the case of an increased tensile load of the material.

It is an object of the present invention to provide a method for producing a prosthetic glove and a prosthetic glove, by way of which the mobility of a prosthetic hand is restricted as little as possible.

This object is achieved by a method having the features of the main claim, and by a prosthetic glove having the features of the coordinate claim. Advantageous embodiments and refinements of the invention are disclosed in the dependent claims, the description and the figures.

The method for producing a prosthetic glove, comprising the steps of providing a first textile blank having a dorsum of hand surface, the so-called dorsum manus, and finger portions that extend away from the dorsum of hand surface; providing a second textile blank having a palm of hand surface, the so-called palma manus, and finger portions that extend away from the palm of hand surface, wherein at least the finger portions of the textile blanks are of an elastic design or comprise elastic regions; arranging the two textile blanks in such a manner that correspondingly designed finger portions are assigned to one another, in particular lie opposite one another; and connecting the two textile blanks directly to one another along the longitudinal extent of the finger portions on the peripheral regions of the latter so as to form finger sleeves, provides that in a relaxed state at least one finger portion of the second textile blank is shorter than the opposite assigned finger portion of the first textile blank, and the finger portion of the second textile blank is stretched when connecting the finger portions. The stretching of the finger portion or of the finger portions of the second textile blank that are shorter than the respective opposite finger portion of the first textile blank, takes place during manufacturing and during joining, in particular while connecting the two textile blanks in the peripheral regions of the latter. The stretching of the finger portion of the second textile blank advantageously takes place to the length of the non-stretched finger portion of the first textile blank that is to be sewn to form a finger sleeve. The stretching or elongation of the second textile blank, or of the corresponding finger portion, takes place when connecting the two textile blanks on the peripheral regions of the finger portions that are connected so as to form the finger sleeves. The second, inner textile blank is elongated when being connected, in particular when being stitched, adhesively bonded or welded, so that the peripheral region has the same length as the peripheral region of the non-elongated textile blank, thus that of the outer hand surface. Owing to the stretching or elongating of the second textile blank at least in the region of the connected finger portions, the corresponding finger, or the corresponding finger sleeve is subjected to a preliminary flexion due to the difference in length in the elastic materials in the non-elongated state, so that a comparatively minor loss of gripping force occurs in a worn prosthetic glove.

In one refinement, all finger portions of the second textile blank are shorter than the correspondingly designed finger portions of the first textile blank and are accordingly stretched when being connected to the first textile blank. As a result, all fingers are subjected to a preliminary flexion so that the entire prosthetic glove supports a flexion motion or at least impedes the latter to a lesser extent, and a position of the prosthetic hand which comes close to the natural position of the hand is supported. If a prosthetic hand is actuated in such a way that all fingers are extended, stretching of the material of the second textile blank occurs in the event of the prosthetic fingers being stretched to the maximum. During complete flexion of the prosthetic fingers, stretching of the material of the prosthetic glove occurs on the side of the back of the hand in such a way that a uniform load on the material, proceeding from the natural position of the hand with slightly flexed fingers, can be achieved by flexion and extension.

In one refinement, at least one finger portion of the second textile blank is narrower than the correspondingly designed finger portions of the first textile blank, and the finger portions are connected to one another at a base point between two finger portions on the dorsum of hand surface and the palm of hand surface. It is prevented in this way that the connection point or the connecting seam between the two textile blanks lies between two fingers. The second textile blank, at least in the finger region on the palm of the hand side, forms a smooth shape which is not interrupted by any seam. The connecting seam, which in a prosthetic glove with fingertips surrounds the entire palm of hand surface on the circumference and when viewed from above onto the palm of the hand side of a flat hand follows the contour, prevents blocking of the fingers when gripping. The material of the first textile blank is placed from the dorsum of the hand laterally over the prosthetic finger or prosthetic fingers and then connected to the first textile blank. The two-part prosthetic glove here is made in such a way that not only the length of the inner textile blank is stretched during manufacturing, but also the longer finger portion, which is not stretched in the longitudinal extent, is folded and positioned in such a way that a prosthetic finger is laterally completely covered by the first textile blank. In the finger sleeve without a fingertip, the first textile blank in the finger portion is bent in a U-shaped manner when viewed in the cross section; the second finger portion forms a substantially straight connection between the two free legs. The connection, in particular the connecting seam, advantageously does not run between the digits.

In one refinement, the textile blanks are made of an elastic material, in particular of a bi-elastic material, such that an elastic deformation is possible not only in the longitudinal extent of the finger portion, thus from the base area on the dorsum of the hand side or palm of the hand side away in the direction of proximal to distal, but also transversely thereto. Apart from a completely elastic embodiment of the finger portions, the latter can also be designed so as to be only partially elastic. Moreover, the region of the palm of hand surface, or the region of the dorsum of hand surface, can also be of an elastic design, or be designed to be elastic in the regions. The elasticity can be unidirectional as well as bidirectional; the embodiment of the elasticity in the regions can also be unidirectional or bidirectional; the embodiment of the elasticity in regions can also be unidirectional or bidirectional.

In one embodiment, at least one finger sleeve is open at the distal end when the two textile finger portions have been connected to one another. Subsequently an end cap is fastened to the finger sleeve or fingers sleeves open at the distal end, so as to form a closed prosthetic glove. The end cap can be adhesively bonded, welded, or sewn thereto. In one advantageous embodiment, the end cap is formed from a non-textile material, for example from an elastomer.

Alternatively, it is provided that two finger portions of the textile blanks are connected to one another so as to form a finger sleeve with a closed fingertip. Here, all prosthetic fingers or only one prosthetic finger can consist exclusively of the textile finger portions with a closed fingertip. Additionally, there is the possibility that the closed finger sleeves with fingertips that are formed by the two textile blanks are coated on the external side, for example provided with an elastomer in the region of the fingertip. Coatings or reinforcements can also be disposed on or applied to other regions of the prosthetic glove.

A prosthetic glove having a first textile blank having a dorsum of hand surface and finger portions that extend away from the dorsum of hand surface, a second textile blank having a palm of hand surface and finger portions that extend away from the palm of hand surface, wherein at least the finger portions of the textile blanks are of an elastic design or comprise elastic portions, and correspondingly designed finger portions lie opposite one another, provides that the two textile blanks are connected directly to one another along the longitudinal extent of the finger portions on the peripheral regions of the latter, form finger sleeves, and in a relaxed non-connected state at least one finger portion of the second textile blank is shorter than the opposite finger portion of the first textile blank. A preliminary flexion results due to the elastic embodiment of the material of the finger portions, in particular of the material of the finger portion or of the finger portions of the second textile blank, because the finger portion lying on the inside is stretched or elongated in order for the finger portions to be connected. The stretching or elongating advantageously takes place to the same length as the non-stretched first finger portion, so that a straight finger termination or front distal finger periphery results in the event of a finger sleeve without a fingertip. If a fingertip is formed, the finger portion of the dorsum of the hand or of the first textile blank is guided in the distal direction in such a way that the connecting seam does not lie in the region between the digits and advantageously does not form the distal end termination of the finger or of the finger sleeve.

In one embodiment of the prosthetic glove, all finger portions of the second textile blank are shorter than the correspondingly designed and arranged finger portions of the first textile blank, so that a preliminary flexion after manufacturing and in the non-worn state results for all fingers.

At least one finger portion of the second textile blank can be narrower than the correspondingly designed finger portion of the first textile blank, so that it is prevented that internal seams or connections are present in the intermediate region between fingers or between digits. As a result, it is prevented that material accumulations are positioned between the prosthetic fingers when gripping with or closing the prosthetic hand. The closing of the hand can be facilitated as a result.

The textile blanks are connected to one another by way of at least one connecting seam, wherein the connecting seam can be embodied as a stitched seam, an adhesively bonded seam, a welded seam or any other thermal connection, for example a thermal compression. The connecting seam connects the two textile blanks directly to one another in such a way that the finger sleeve and the remaining part of the prosthetic glove are formed in particular in the region of the finger portions by the two textile blanks.

In one embodiment of the prosthetic glove, the connecting seam is formed in an encircling manner while omitting a proximal introduction opening, so that the complete prosthetic glove can be manufactured by way of a single seam. The proximal introduction opening serves to introduce the prosthetic hand into the prosthetic glove. If a plurality of connecting seams are used in order to connect the two textile blanks to one another, it is provided in one embodiment that the textile blanks are connected to one another by way of a plurality of connecting seams while omitting a proximal introduction opening and at least one distal finger opening on the finger portions. In this instance, the prosthetic glove does not have any fingertip on at least one finger, so that the respective connecting seam on the finger sleeve terminates on the free end. The connecting seam can be formed as a stitched seam, welded seam, adhesively bonded seam or any other linear permanent connection. A linear connection can also be formed by a linear arrangement of punctiform connections, for example spot welds, adhesive spots, or any other connection or a combination thereof.

The at least one connecting seam between two finger sleeves runs on the inside of the palm of the hand in such a way that the region between the digits remains free of double material or seam material.

In one embodiment, the textile blanks between two finger portions have a proximal intermediate region in which the textile blanks are connected to one another at a base point. The first textile blank and the second textile blank are coupled to one another at the finger root in such a way that the finger sleeves are separated from one another.

In one embodiment, the finger portions for the index finger, the middle finger, the ring finger and the little finger are designed with the same orientation on the textile blanks in such a way that said finger portions are identically shaped with the exception of a different size in the length and optionally in the width. The textile blank for the inner palm of the hand is smaller and shorter than the textile blank for the dorsum of the hand and the lateral regions of the finger sleeves. The palm of hand surface from which the finger portions extend away can also be smaller here than the region of the dorsum of the hand in such a way that the inner, first textile blank is elongated during connecting or stitching, so that the seam lengths correspond to the peripheral regions of the non-elongated first textile blank. For this purpose, the elastic material of the second textile blank is elongated in such a way that the lengths of the peripheral regions correspond to one another when connecting the textile blanks.

In one embodiment, the longitudinal extent of the finger portion for the thumb on the first textile blank has a different orientation than the orientation of the longitudinal extent of the finger portion for the thumb of the second textile blank. In particular, the finger portion for the thumb of the first textile blank extends at a smaller angle in relation to the longitudinal extent of the finger portion of the index finger than the finger portion of the second textile blank. The orientations of the longitudinal extent between the thumb and the index finger in the first textile blank is 30° to 60°, for example, while said orientation is between 70° and 90° in the second textile blank. As a result, apart from the preliminary flexion due to the longitudinal difference, an orientation of the thumb of the prosthetic glove which is adapted to the different directions of movement of the prosthetic thumb is also effected. As a result, the connecting seam in the region of the thumb is repositioned inwardly, in the direction of the thenar eminence so that a convergence of thumb and finger when performing a pincer grip does not lead to a compression of material in the region of the connecting seam.

In one embodiment of the prosthetic glove, at least one finger sleeve has an open distal end to which is fastened an end cap in such a way that a closed finger sleeve with a fingertip is present. The end cap is in particular formed from a different material than the textile blank or the textile blanks, in particular as a cap of an elastomer, so as to dispose a stable and resistant material in those regions of the prosthetic hand that are particularly subjected to wear. Alternatively, at least one finger is designed in such a way that the mutually opposite finger portions of the textile blanks are connected so as to form a finger sleeve with a closed fingertip, so that the entire finger sleeve is composed of the material of the textile blanks.

Exemplary embodiments of the invention will be explained in more detail hereunder with reference to the figures in which:

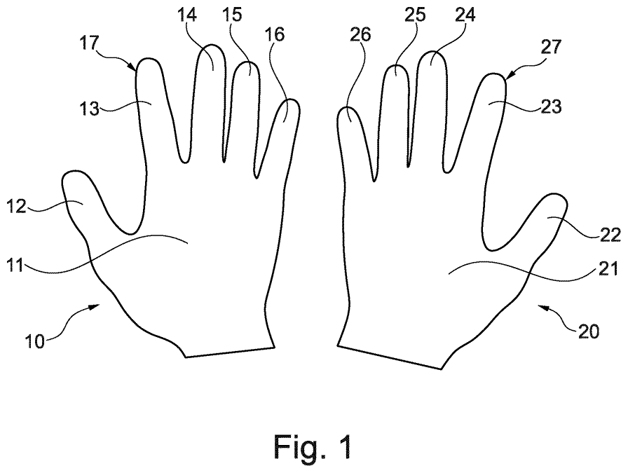

FIG. 1 shows a cutting pattern of a textile glove according to the prior art;

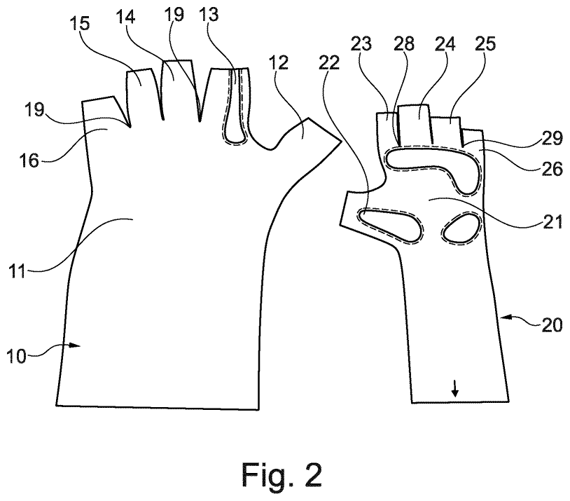

FIG. 2 shows an illustration of two textile blanks;

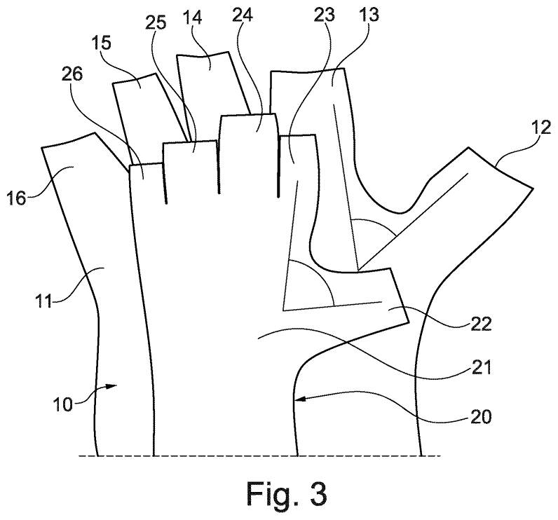

FIG. 3 shows an illustration of two textile blanks placed on top of one another; and

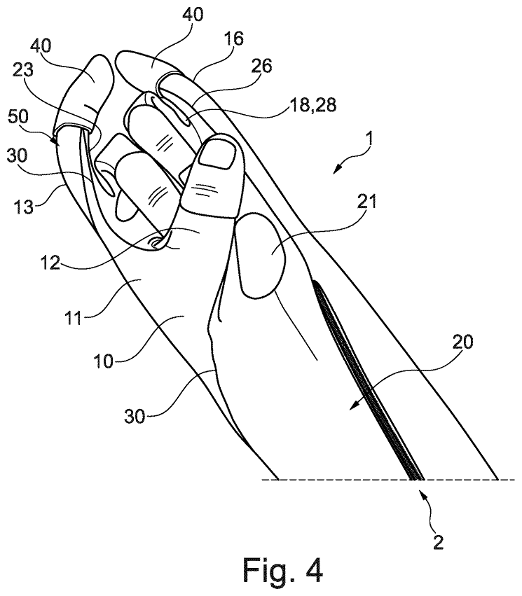

FIG. 4 shows a completely sewn prosthetic glove with end caps.

FIG. 1 shows two textile blanks according to the prior art for gloves, which have the same shape. The two textile blanks 10, 20 have a dorsum of hand surface 11 and a palm of hand surface 21 from which finger regions 12, 13, 14, 15, 16, 22, 23, 24, 25, 26 extend outward toward the distal end. For producing a glove, the two blanks are placed on top of one another and sewn to one another by way of a circumferential seam in a peripheral region 17 of the textile blanks 10, 20. In this way, a closed glove having a proximal introduction opening which is formed on the non-connected ends of the textile blanks 10, 20 that lie opposite the fingers is formed.

The textile blanks 10, 20 according to the prosthetic glove according to the invention are illustrated in FIG. 2. The left illustration shows the textile blank 10 for the upper side of the prosthetic glove, having a dorsum of hand surface 11 and finger portions 12 to 16 which extend away from the latter in the distal direction. In the exemplary embodiment illustrated, the finger portions 12 to 16 are not formed to be so long that they cover the complete prosthetic finger in the longitudinal extent, but are only so long that the tips of the prosthetic fingers protrude from the prosthetic glove. A proximal intermediate region 19, which is formed in the region of the finger root or of the transition from a main body of the prosthetic hand, to which the prosthetic fingers are fastened in an articulated manner, to the prosthetic finger, is in each case formed between two finger portions.

The second textile blank 20 for the inside of the prosthetic glove, having a palm of hand surface 21, is illustrated in the right-hand illustration of FIG. 3. This textile blank 20 also has five finger portions 22 to 26 between each of which is formed a proximal intermediate region 29 of which only one is plotted for reasons of clarity. It can already be seen from FIG. 2 that the second textile blank 20 is smaller than the first textile blank 10. The palm of hand surface 21 as well as the finger portions 22 to 26 are narrower and shorter than the corresponding regions of the first textile blank 10. It can likewise be derived from FIG. 2 that the orientation of the longitudinal extent of the finger portion 22 for the thumb of the second textile blank 20 has a different orientation relative to the longitudinal extent of the index finger portion 23 than the longitudinal orientation of the finger portion 12 of the thumb of the first textile blank 10 relative to the longitudinal extent of the finger portion 13 of the index finger. In the exemplary embodiment, the longitudinal extents in the finger portions 23 to 26 of the second textile blank 20 are substantially oriented in parallel, while the longitudinal extents of the finger portions 13 to 16 of the first textile blank 10 are slightly spread apart but likewise run fundamentally in the proximal-distal direction.

The difference in size between the two textile blanks 10, 20 becomes evident by means of FIG. 3. In the exemplary embodiment, the first textile blank 10 is almost double the size of the second textile blank 20. The width of the finger portions 23 to 26 is less than the width of the finger portions 13 to 16 of the first textile blank 10; likewise, the length of the finger portions 22 to 26 of the second textile blank 20 is substantially less than the length of the finger portions 12 to 16 of the first textile blank 10, in each case measured in the non-elongated state. Likewise, the difference in terms of orientation of the finger portions 12, 22 for the thumb is highlighted; the angle between the longitudinal extent of the index finger 23 in relation to the longitudinal extent of the thumb 22 of the second textile blank 20 is approximately 80°, whereas the angle between the index finger 13 and the thumb 12 of the first blank 10 is approximately 45°. As a result of this different orientation of the longitudinal extent of the thumb portion 12, 22, a movement of the prosthetic thumb in the direction toward the palm of the hand side is facilitated. In order to manufacture a prosthetic glove from the two textile blanks 10, 20, the second textile blank 20 is elongated so far that the peripheral regions to be connected in each case are brought to be congruent with a non-elongated corresponding portion of the first textile blank 10. At this point they are then sewn to one another or permanently connected to one another in another manner, for example adhesively bonded or welded. The length of the connecting seam, when the two textile blanks 10, 20 are sewn to one another, corresponds to the length of the peripheral region of the non-elongated first textile blank 10, along which the connecting seam leads. In the exemplary embodiment illustrated, the finger portions are provided with open ends so that the prosthetic glove cannot be closed by way of a single circumferential seam. Instead, a separate seam has to be provided between the individual finger portions in order to connect the peripheral regions of the individual finger portions directly to one another. When connecting the two textile blanks 10, 20, at least the region of the second textile blank 20 that is being sewn is in an elongated state. In the exemplary embodiment illustrated, the material of the second textile blank 20 is bielastic and can be elastically elongated in the longitudinal extent as well as transversely thereto. Alternatively, there is only an elasticity in the longitudinal extent from proximal to distal, as a result of which a preliminary flexion is provided when the prosthetic glove is not worn.

A prosthetic glove 1 is shown in a completed state in FIG. 4. The prosthetic glove 1 has a proximal introduction opening 2 which enables a prosthetic hand not illustrated to be introduced. The prosthetic glove 1 can also have a zip fastener or any other closure device for facilitating the introduction of a prosthetic hand. The two textile blanks 10, 20 are designed according to the blanks of FIGS. 2 and 3, so that there are a plurality of connecting seams 30 in order to fixedly establish the textile blanks 10, 20 directly on one another in their peripheral regions. As a result of the textile blanks 10, 20 being connected on the peripheral regions while the second textile blank 20 is elongated, a preliminary flexion which can be seen on the prosthetic glove 1 results by virtue of the elastic restoring forces of the shorter embodiment of the non-elongated second textile blank 20. All fingers which have been sewn to form finger sleeves 50 with open ends have a preliminary flexion or curvature toward the inside. The finger sleeves 50 of all fingers are not produced completely from one textile material, but have end caps 40 which are produced from another material, presently an elastomer, and have been subsequently fixed to the open finger ends. The end caps 40 can be adhesively bonded, welded and/or sewn thereto. The textile blanks 10, 20 are connected to one another in each case at a base point 18, 28 in the region of the proximal intermediate regions 19, 29 in such a way that the finger sleeves 50 of the individual finger portions are separated from one another. The seams 30 do not run between the finger regions but lie on the lower side of the prosthetic glove 1.

### LIST OF REFERENCE SIGNS

1 Prosthetic glove
2 Introduction opening
10 Textile blank
11 Dorsum of hand surface
12 Thumb
13 Index finger
14 Middle finger
15 Ring finger
16 Little finger
17 Peripheral region
18 Base point
19 Intermediate region
20 Textile blank
21 Palm of hand surface
22 Thumb
23 Index finger
24 Middle finger
25 Ring finger
26 Little finger
28 Base point
29 Intermediate region
30 Seam
40 End caps
50 Finger sleeve

1. A method for producing a prosthetic glove,
comprising:
providing a first textile blank comprising a dorsum of hand surface and finger portions that extend away from the dorsum of hand surface;
providing a second textile blank comprising a palm of hand surface and finger portions that extend away from the palm of hand surface, wherein at least the finger portions of the first textile blank and the finger portions of the second textile blank are of an elastic design or comprise elastic regions;
arranging the first textile blank and the second textile blank such that correspondingly designed finger portions of the first textile bland and the finger portions of the second textile blank are assigned to one another;
connecting the first textile blank and the second textile blank directly to one another along a longitudinal extent at a peripheral region of the finger portions of the first textile blank and a longitudinal extent at a peripheral region of the finger portions of the second textile blank to form finger sleeves, wherein in a relaxed state at least one finger portion of the second textile blank is shorter than an opposite assigned finger portion of the first textile blank, and the at least one finger portion of the second textile blank is stretched when being connected.

2. The method as claimed in claim 1, wherein
all finger portions of the second textile blank are shorter than all of the corresponding finger portions of the first textile blank, and wherein all finger portions of the second textile blank are stretched when being connected to the first textile blank.

3. The method as claimed in claim 1, wherein at least one finger portion of the second textile blank is narrower than a corresponding finger portion of the first textile blank, and wherein the finger portions of the first textile blank and the finger portions of the second textile blank are connected to one another at a base point between two finger portions on the dorsum of hand surface and the palm of hand surface.

4. The method as claimed in claim 1 wherein both the first textile blank and the second textile blank are composed of an elastic material.

5. The method as claimed in claim 1 wherein at least one finger sleeve is open at a distal end, and an end cap is fastened to the at least one finger sleeve.

6. The method as claimed in claim 1 wherein at least one finger portion of the first textile blank and/or at least one finger portion of the second textile blank is connected so as to form a finger sleeve with a closed fingertip.

7. A prosthetic glove, comprising:
a first textile blank comprising a dorsum of hand surface and finger portions that extend away from the dorsum of hand surface;
a second textile blank comprising a palm of hand surface and finger portions that extend away from the palm of hand surface,
wherein at least the finger portions of the first textile blank and the finger portions of the second textile blank are of an elastic design or comprise elastic portions, and
wherein correspondingly designed finger portions of the first textile blank and finger portions of the second textile blank lie opposite one another,
wherein the first textile blank and the second textile blank are connected directly to one another along a longitudinal extent at a peripheral region of the finger portions of the first textile blank and at a longitudinal extent at a peripheral region of the second textile blank to form a plurality of finger sleeves, and
wherein in a relaxed non-connected state at least one finger portion of the second textile blank is shorter than an opposite finger portion of the first textile blank.

8. The prosthetic glove as claimed in claim 7, wherein all finger portions of the second textile blank are shorter than all of the corresponding finger portions of the first textile blank.

9. The prosthetic glove as claimed in claim 7, wherein at least one finger portion of the second textile blank is narrower than a corresponding finger portion of the first textile blank.

10. The prosthetic glove as claimed in claim 7 wherein both the first textile blank and the second textile blank are composed of an elastic material.

11. The prosthetic glove as claimed in claim 7 wherein the palm of hand surface is narrower and/or shorter than the dorsum of hand surface.

12. The prosthetic glove as claimed in claim 7 wherein the first textile blank and the second textile blank are connected by at least one connecting seam.

13. The prosthetic glove as claimed in claim 12, wherein the at least one connecting seam is formed in an encircling manner while omitting a proximal introduction opening.

14. The prosthetic glove as claimed in claim 7 wherein the first textile blank and the second textile blank are connected by a plurality of connecting seams while omitting a proximal introduction opening and at least one distal finger opening.

15. The prosthetic glove as claimed in claim 12 wherein the at least one connecting seam runs between two finger sleeves of the plurality of finger sleeves on an inside of the palm of hand surface.

16. The prosthetic glove as claimed in claim 7 wherein the first textile blank and the second textile blank between two finger portions have a proximal intermediate region in which the first textile blank and the second textile blank are connected to one another at a base point.

17. The prosthetic glove as claimed in claim 7 wherein the finger portions of the first textile blank and the finger portions of the second textile blank are designed for an index finger, a middle finger, a ring finger, and a little finger, and are designed with an identical orientation on the first textile blank and the second textile blank.

18. The prosthetic glove as claimed in claim 7 wherein the longitudinal extent of the finger portion corresponding with at least a thumb on the first textile blank has a different orientation than the longitudinal extent of the finger portion corresponding with at least the thumb of the second textile blank.

19. The prosthetic glove as claimed in claim 7 wherein at least one finger sleeve of the plurality of finger sleeves is designed with an open distal end to which is fastened an end cap.

20. The prosthetic glove as claimed in claim 7 wherein the first textile blank and the second textile blank each comprise at least one opposite finger portion connected to form a finger sleeve of the plurality of finger sleeves with a closed fingertip.
